(19) United States
(12) Patent Application Publication
(10) Pub. No.: US 2015/0299185 A1
(43) Pub. Date: Oct. 22, 2015

(54) NOVEL IMIDAZOPYRIDINE DERIVATIVES AS A TYROSINE KINASE INHIBITOR

(71) Applicant: HANMI PHARM. CO., LTD., Hwaseong-si, Gyeonggi-do (KR)

(72) Inventors: Kyung Ik Lee, Anyang-si (KR); Jae Yi Sim, Seoul (KR); Ho Seok Kim, Hwaseong-si (KR); Tae Hee Ha, Hwaseong-si (KR); Kwee Hyun Suh, Suwon-si (KR)

(73) Assignee: HANMI PHARM. CO., LTD., Hwaseong-si, Gyeonggi-do (KR)

(21) Appl. No.: 14/368,702

(22) PCT Filed: Dec. 27, 2012
(86) PCT No.: PCT/KR2012/011570

§ 371 Date: Jun. 25, 2014

(30) Foreign Application Priority Data
Dec. 28, 2011 (KR) 10-2011-0144450

(51) Int. Cl. C07D 471/04; A61K 31/437; A61K 31/4545; A61K 31/5377; A61K 31/496; A61K 45/06

(52) U.S. Cl. CPC C07D 471/04; A61K 31/496; A61K 45/06; A61K 31/4545; A61K 31/5377; A61K 31/437

(57) ABSTRACT

Provided is a novel imidazopyridine derivative having irreversible tyrosine kinase inhibiting activities, and a pharmaceutical composition comprising the same which can be useful for prevention or treatment of inflammatory diseases, autoimmune diseases, proliferative diseases or hyperproliferative diseases, immunologically mediated diseases, cancers or tumors.

### FIELD OF THE INVENTION

The present invention relates to novel imidazopyridine derivatives having irreversible tyrosine kinase inhibiting activities, and pharmaceutical compositions comprising the same as an active ingredient.

### BACKGROUND OF THE INVENTION

A protein kinase is an enzyme that modifies other proteins by chemically adding phosphate groups to a specific residue thereof via phosphorylation. The human genome contains about 500 protein kinase genes and they constitute about 2% of all human genes. In general, protein kinases can be classified into three types depending on their substrates: serine/threonine-specific protein kinases which phosphorylate serine and/or threonine residues, tyrosine-specific protein kinases which phosphorylate tyrosine residues and protein kinases which phosphorylate tyrosine and serine/threonine residues. Protein kinases play a key role in mediation of signal transduction from the cell surface to the nucleus in response to a variety of extracellular stimuli. They regulate several physiological and pathological cellular phenomena, including cell division, proliferation, differentiation, apoptosis, cell mobility, mitogenesis, etc., and hence they are closely related with various diseases. Examples of such kinase-related diseases are: autoimmune disorders such as atopic dermatitis, asthma, rheumatoid arthritis, Crohn's disease, psoriasis, Crouzon syndrome, achondroplasia, and thanatophoric dysplasia; cancer such as prostate cancer, colorectal cancer, breast cancer, brain and throat cancer, leukemia and lymphoma; diabetes; restenosis; atherosclerosis; renal and hepatic fibrosis; myeloproliferative disorder and lymphoproliferative disorder; and eye disease. It is known that such diseases are caused directly or indirectly by interruption in kinase regulating mechanism such as mutation, overexpression or abnormal activation of kinase enzyme, and overproduction or underproduction of growth factors or cytokines which affect up-stream or down-stream signaling. Therefore, it is expected that such diseases may be prevented or treated by selectively inhibiting the mechanism of kinase, and thus various attempts have been made to discover an effective protein kinase inhibitor in the fields of medicine and chemistry.

Meanwhile, inflammation is a cause of disease such as rheumatoid arthritis, etc. Continuous attempts have been made to develop an effective medicine to treat inflammation despite the recent discovery of biological treatments. Various evidences have been found which support T-cells (or T-lymphocytes) and B-cells (or B-lymphocytes) play an important role in connection with the outbreak of inflammatory diseases, autoimmune diseases, proliferative or hyper-proliferative diseases and/or immunologically mediated diseases.

Such T-cells mediate signal transduction by receiving signals from antigen presenting cells through T-cell receptor (TCR) located on the surface of the cell which activates various kinases such as Janus kinase (JAK) so as to forward the signal to effectors. In this regard, JAK proteins, as tyrosine kinases, may be activated by hematopoietic cytokine as well as interferon, and this process can regulate the activation of transcriptional regulators, STAT proteins. Therapeutic possibilities based on the inhibition (or promotion) of JAK/STAT pathway may provide a potent medication in the field of immunomodulation.

Among 4 types of JAK proteins, JAK3 is believed to be implicated in inflammation as it is expressed only in T-cells and activated by IL-2. Unlike JAK2 which participates in hemopoietic activity and red blood cell homeostasis or JAK1 which can be expressed in different types of tissues, JAK3 is mostly expressed in lymphocytes and plays very important role in signaling by using various cytokines including IL-2, IL-4, IL-7, IL-9, IL-15 and the like, and therefore JAK3 is getting attention in respect of side effects (Flanagan et al., Journal of Medicinal Chemistry, 53, 8468, 2010). According to animal studies, JAK3 plays important role not only in maturation of B-cells and T-cells, but also in maintenance of functions of T-cells. Therefore, a JAK inhibitor, especially JAK3 inhibitor, may be useful for treatment of autoimmune disorders such as rheumatoid arthritis, psoriasis, atopic dermatitis, lupus, multiple sclerosis, Type I diabetes and diabetic complications, cancer, asthma, thyroid autoimmune disease, ulcerative colitis, Crohn's disease, Alzheimer's disease, leukemia, etc. as well as various conditions where immunosuppression is required such as allograft rejection and xenotransplantation (Pesu M, Laurence A, Kishore N, et al., Immunol. Rev, 223, 132, 2008; Kawahara A, Minami Y, Miyazaki T, et al., Proc. Natl. Acad. Sci. USA, 92, 8724, 1995; Nosaka T, van Deursen J M A, Tripp R A, et al., Science, 270, 800, 1995; Papageorgiou A C, Wikman L E K., et al., Trends Pharm. Sci., 25, 558, 2004)

Meanwhile, Bruton's tyrosine kinase (BTK) is a type of TEC-kinase family which plays an important role in activation of B-cells as well as signal transduction. In 1993, it was discovered that mutations in BTK are related with the major B-cell immune deficiency, X-linked Agammaglobulinemia (XLA) and mouse X-linked immunodeficiency (XID).

BTK is a key regulator of B-cell development, activation, signaling, and survival (Kurosaki, Curr. Op. Imm., 276-281, 2000; Schaeffer and Schwartzberg, Curr. Op. Imm., 282-288, 2000]. In addition, BTK plays a role in a number of other hematopoietic cell signaling pathways, e.g., toll-like receptor (TLR)- and cytokine receptor-mediated TNF-α production in macrophages, IgE receptor (FcepsilonRi) signaling in mast cells, inhibition of Fas/APO-1 apoptotic signaling in B-lymphocytes, and collagen-stimulated platelet aggregation.

BTK participates in signal transduction pathways initiated by the binding of a variety of extracellular ligands to their cell surface receptors. After B-cell antigen receptor (BCR) ligation by antigen, BTK activation by the concerted actions of protein tyrosine kinases Lyn and Syk is required for induction of phospholipase C-γ2-mediated calcium mobilization (Kurosaki, T., Curr. Opin. Immunol., 9, 309-318, 1997). Therefore, inhibition of BTK can become a useful therapeutic option since it prevents the development of B-cell mediated diseases.

For instance, BTK deficient mice have been shown to be resistant to disease manifestations in collagen-induced arthritis, and BTK inhibitor is known to be effective against collagen-induced arthritis in mice dose-dependently (Jansson and Holmdahl, Clin. Exp. Immunol., 94, 459, 1993; Pan et al., Chem. Med Chem., 2, 58, 2007). Therefore, an effective BTK inhibitor may be useful for treatment of rheumatoid arthritis.

Further, inhibition of BTK activation can be useful for treatment of autoimmune disease and/or inflammatory disease and/or allergic disease, e.g., systemic lupus erythematosus (SLE), rheumatoid arthritis, psoriatic arthritis, osteoarthritis, juvenile arthritis, diabetes, myasthenia gravis, Hashimoto's thyroiditis, multiple sclerosis, ankylosing spondylitis, angiitis, inflammatory bowel disease, psoriasis, alopecia universalis, idiopathic thrombocytopenic purpura (ITP), allergy, allergic conjunctivitis, allergic rhinitis, atopic dermatitis, and asthma, but not limited thereto. Also, it is known that BTK regulates apoptosis in cells, hence inhibition of BTK activation can be used to treat B-cell lymphoma and leukemia as well.

As explained above, the Janus kinase such as JAK3 and TEC kinase such as BTK play important roles in activation of T-cell and/or B-cell that are closely related with development of inflammatory diseases, autoimmune diseases, proliferative diseases or hyperproferative diseases and immunologically mediated diseases. Hence, development of an effective inhibitor of such kinases may lead to discovery of potent drug for treatment of various inflammatory diseases, autoimmune diseases, proliferative diseases or hyperproliferative diseases, and immunologically mediated diseases.

Currently, Tofacitinib (CP-690550), an oral drug, as an inhibitor of JAK3, is in development by Pfizer and a phase III trial is under way. PCI-32765 (Pharmacyclics), as an inhibitor of BTK, is in phase I clinical trial stage; however, it has been reported that the drug could activate a different target accompanied by adverse side effects including skin rash and diarrhea. Therefore, there is a strong need for a novel drug which can inhibit Janus kinase and TEC kinase in a safe and effective manner.

### SUMMARY OF THE INVENTION

Therefore, it is an object of the present invention to provide a novel compound which inhibits kinases that are mostly expressed in aberrantly activated lymphocytes (T-lymphocytes and/or B-lymphocytes) including Janus kinase such as JAK3 as well as TEC kinase such as BTK (Burton's tyrosine kinase), ITK (IL2-inducible T-cell kinase), BMX (bone marrow tyrosine kinase), RLK (resting lymphocyte kinase) and the like.

It is another object of the present invention to provide a pharmaceutical composition comprising the inventive compound for prevention or treatment of inflammatory diseases, autoimmune diseases, proliferative diseases or hyperproliferative diseases, immunologically mediated diseases, cancers or tumors.

It is further object of the present invention to provide a method for prevention and treatment of inflammatory diseases, autoimmune diseases, proliferative diseases or hyperproliferative diseases, immunologically mediated diseases, cancers or tumors by using the compound.

In accordance with one aspect of the present invention, there is provided a compound of formula (I), or a pharmaceutically acceptable salt thereof:

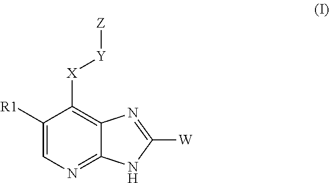

wherein,

R1 is hydrogen, halogen or CN;

X is O, NH, CH₂, S, SO or SO₂;

Y is phenyl or pyridyl;

Z is

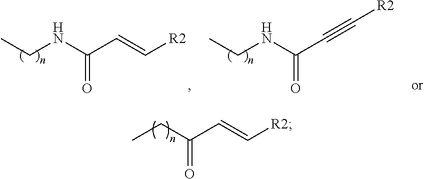

n is an integer ranging from 0 to 4;

R2 is each independently hydrogen, C₁₋₆alkoxy or di(C₁₋₆alkyl)aminomethyl; and

W is phenyl or pyridyl substituted with one or more substituents selected from the group consisting of hydrogen, halogen, hydroxy, nitro, C₁₋₆alkoxy, C₁₋₆alkoxycarbonyl, amino, C₁₋₆alkylamino, C₁₋₆alkylheterocycleamino, carbamoyl, C₁₋₆alkylcarbamoyl, di(C₁₋₆alkyl)carbamoyl, C₁₋₆alkylheterocyclecarbamoyl, C₁₋₆alkylheterocycleC₁₋₆alkyl, sulfamoyl, C₁₋₆alkylsulfanyl, C₁₋₆alkylsulfonyl, C₁₋₆alkylsulfinyl, C₁₋₆alkoxyC₁₋₆alkyl, C₁₋₆alkoxyC₁₋₆alkoxy, di(C₁₋₆alkyl)amino, di(C₁₋₆alkyl)aminoC₁₋₆alkyl, di(C₁₋₆alkyl)aminoC₁₋₆alkoxy, carboxyl, heterocycle, C₁₋₆alkylheterocycle, hydroxyheterocycle, hydroxyC₁₋₆alkylheterocycle, C₁₋₆alkoxyC₁₋₆alkylheterocycle, heterocyclic-oxy, heterocyclicC₁₋₆alkyl, heterocyclicaminoC₁₋₆alkyl, heterocycliccarbonyl, and heterocyclic-C₁₋₆alkylcarbonyl, wherein the heterocycle is independently a saturated 3- to 8-membered monocyclic hetero ring containing one or more of heteroatoms independently selected from N, O and S.

In accordance with another aspect of the present invention, there is provided a pharmaceutical composition comprising the compound of formula (I) or a pharmaceutically acceptable salt thereof for prevention or treatment of inflammatory diseases, autoimmune diseases, proliferative diseases or hyperproliferative diseases, immunologically mediated diseases, cancers or tumors.

In accordance with further aspect of the present invention, there is provided a method for preventing or treating inflammatory diseases, autoimmune diseases, proliferative diseases or hyperproliferative diseases, immunologically mediated diseases, cancers or tumors in an mammal, comprising the step of administering to the mammal an effective amount of the compound of formula (I) or a pharmaceutically acceptable salt thereof.

The novel imidazopyridine derivatives in accordance with the present invention can selectively and effectively inhibit kinases that are mostly expressed in aberrantly activated lymphocytes (T-lymphocytes and/or B-lymphocytes) including Janus kinase such as JAK3 as well as TEC kinase such as BTK, ITK, BMX and RLK and the like. Therefore, the novel imidazopyridine derivatives as a tyrosine kinase inhibitor in accordance with the present invention may be useful for prevention or treatment of diseases that are mediated by abnormally activated T-lymphocytes, B-lymphocytes or both such as inflammatory diseases, autoimmune diseases, proliferative diseases or hyperproliferative diseases, immunologically mediated diseases, cancers or tumors.

### DETAILED DESCRIPTION OF THE INVENTION

In formula (I), specific examples of the substituent W may be selected from the group consisting of W1 to W30, but not limited thereto.

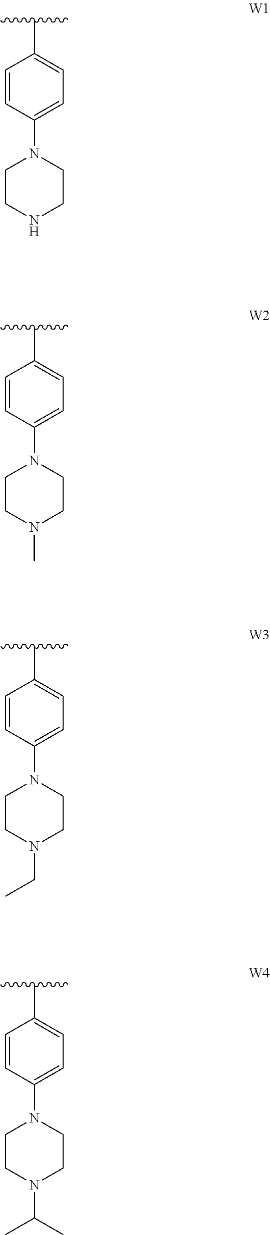 [SMILES: *c1ccc(N2CCNCC2)cc1 ; *c1ccc(N2CCN(C)CC2)cc1 ; *c1ccc(N2CCN(CC)CC2)cc1 ; *c1ccc(N2CCN(C(C)C)CC2)cc1]

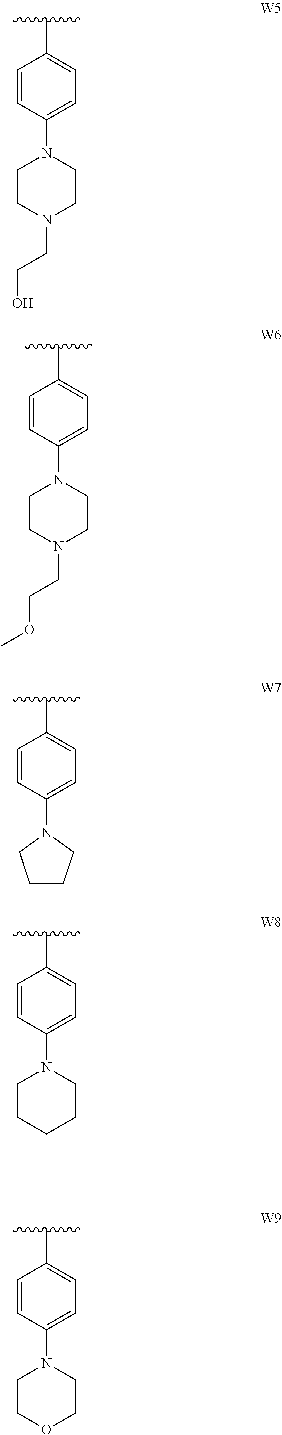 [SMILES: *c1ccc(N2CCN(CCO)CC2)cc1 ; *c1ccc(N2CCN(CCOC)CC2)cc1 ; *c1ccc(N2CCCC2)cc1 ; *c1ccc(N2CCCCC2)cc1 ; *c1ccc(N2CCOCC2)cc1]

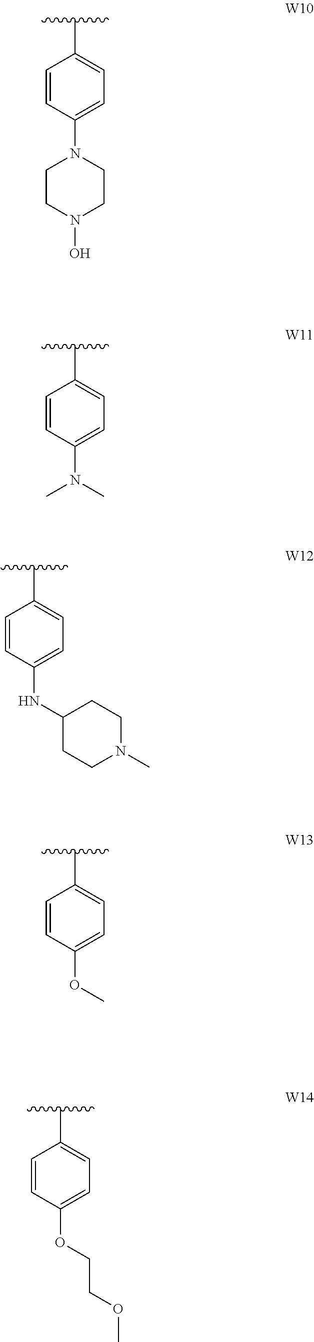

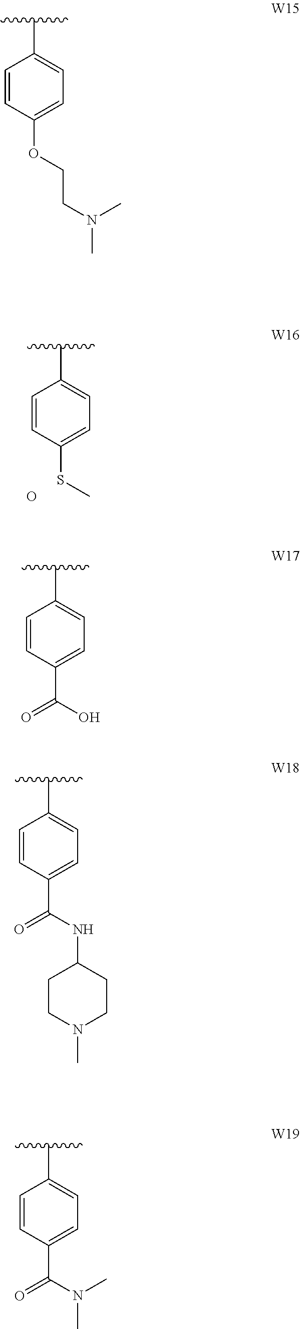

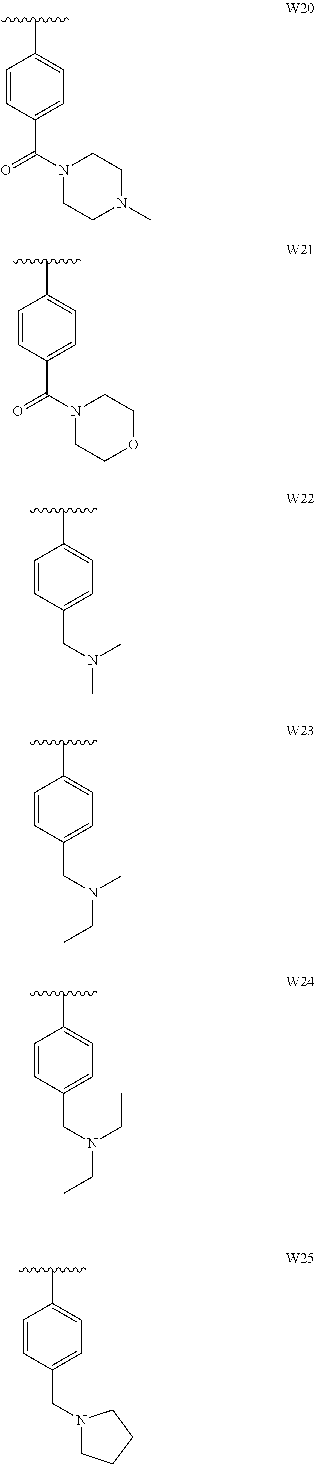 [SMILES: *c1ccc(C(=O)N2CCN(C)CC2)cc1 ; *c1ccc(C(=O)N2CCOCC2)cc1 ; *c1ccc(CN(C)C)cc1 ; *c1ccc(CN(C)CC)cc1 ; *c1ccc(CN(CC)CC)cc1 ; *c1ccc(CN2CCCC2)cc1]

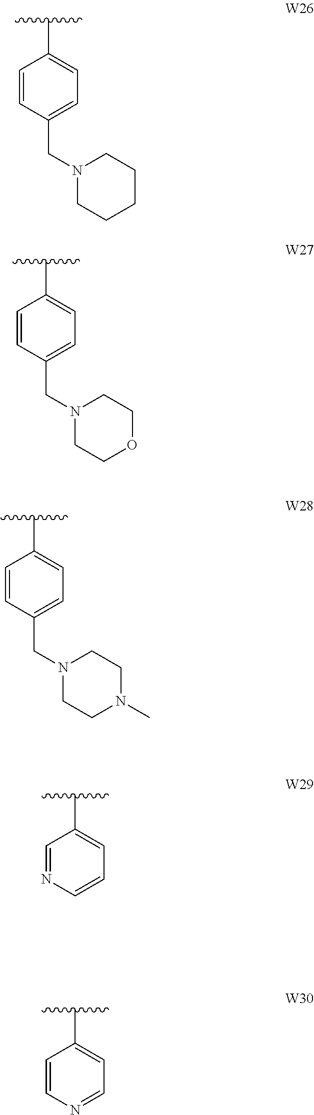 [SMILES: *c1ccc(CN2CCCCC2)cc1 ; *c1ccc(CN2CCOCC2)cc1 ; *c1ccc(CN2CCN(C)CC2)cc1 ; *c1cccnc1 ; *c1ccncc1]

The examples of the compounds in accordance with the present invention are as follows:

N-(3-(6-chloro-2-(4-(4-methylpiperazin-1-yl)phenyl)-3H-imidazo[4,5-b]pyridin-7-yloxy)phenyl)acrylamide;

N-(3-(6-chloro-2-(4-(4-hydroxypiperidin-1-yl)phenyl)-3H-imidazo[4,5-b]pyridin-7-yloxy)phenyl)acrylamide;

N-(3-(6-chloro-2-(4-(4-methylpiperazin-1-carbonyl)phenyl)-3H-imidazo[4,5-b]pyridin-7-yloxy)phenyl)acrylamide;

N-(3-(6-chloro-2-(4-(morpholin-4-carbonyl)phenyl)-3H-imidazo[4,5-b]pyridin-7-yloxy)phenyl)acrylamide;

N-(3-(6-chloro-2-(4-(1-methylpiperidin-4-ylamino)phenyl)-3H-imidazo[4,5-b]pyridin-7-yloxy)phenyl)acrylamide;

N-(3-(6-chloro-2-(4-(dimethylamino)phenyl)-3H-imidazo[4,5-b]pyridin-7-yloxy)phenyl)acrylamide;

N-(3-(6-chloro-2-(4-(methylsulphinyl)phenyl)-3H-imidazo[4,5-b]pyridin-7-yloxy)phenyl)acrylamide;

4-(7-(3-acrylamidophenoxy)-6-chloro-3H-imidazo[4,5-b]pyridin-2-yl)-N,N-dimethylbenzamide;

4-(7-(3-acrylamidophenoxy)-6-chloro-3H-imidazo[4,5-b]pyridin-2-yl)-benzoic acid;

4-(7-(3-acrylamidophenoxy)-6-chloro-3H-imidazo[4,5-b]pyridin-2-yl)-N-(1-methylpiperidin-4-yl)benzamide;

N-(3-(6-chloro-2-(4-((dimethylamino)methyl)phenyl)-3H-imidazo[4,5-b]pyridin-7-yloxy)phenyl)acrylamide;

N-(3-(6-chloro-2-(4-((diethylamino)methyl)phenyl)-3H-imidazo[4,5-b]pyridin-7-yloxy)phenyl)acrylamide;

N-(3-(6-chloro-2-(4-((ethyl(methyl)amino)methyl)phenyl)-3H-imidazo[4,5-b]pyridin-7-yloxy)phenyl)acrylamide;

N-(3-(6-chloro-2-(4-(pyrroldin-1-ylmethyl)phenyl)-3H-imidazo[4,5-b]pyridin-7-yloxy)phenyl)acrylamide;

N-(3-(6-chloro-2-(4-(piperidin-1-ylmethyl)phenyl)-3H-imidazo[4,5-b]pyridin-7-yloxy)phenyl)acrylamide;

N-(3-(6-chloro-2-(4-(morpholinomethyl)phenyl)-3H-imidazo[4,5-b]pyridin-7-yloxy)phenyl)acrylamide;

N-(3-(6-chloro-2-(4-((4-methylpiperazin-1-yl)methyl)phenyl)-3H-imidazo[4,5-b]pyridin-7-yloxy)phenyl)acrylamide;

N-(3-(6-chloro-2-(4-methoxyphenyl)-3H-imidazo[4,5-b]pyridin-7-yloxy)phenyl)acrylamide;

N-(3-(6-chloro-2-(pyridin-4-yl)-3H-imidazo[4,5-b]pyridin-7-yloxy)phenyl)acrylamide; and

N-(3-(2-(4-(4-methylpiperazin-1-yl)phenyl)-3H-imidazo[4,5-b]pyridin-7-yloxy)phenyl)acrylamide.

The compound of formula (I) of the present invention may be prepared by the method shown in Reaction Scheme I as shown below:

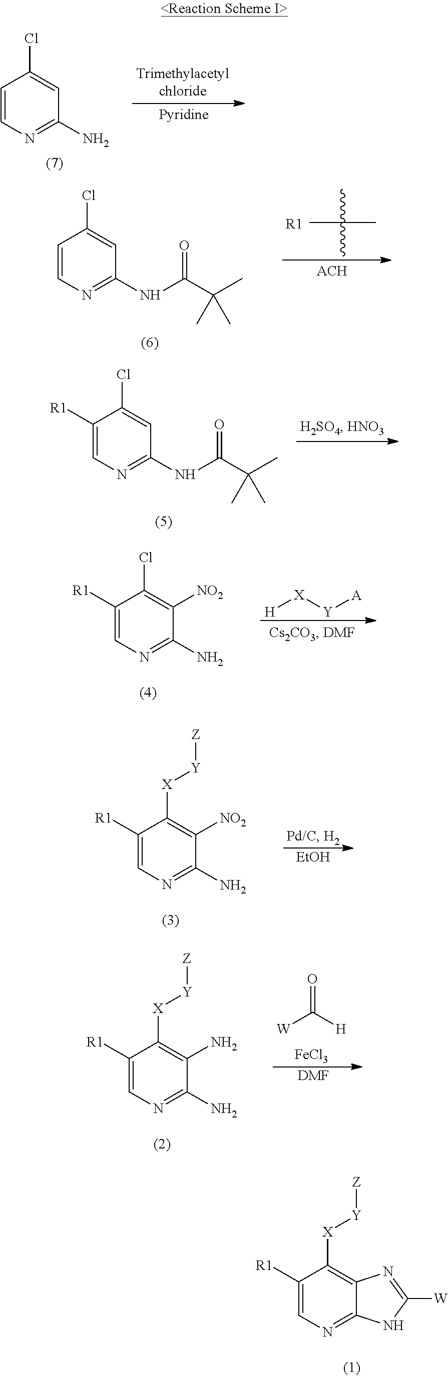

wherein X, Y, Z, W and R1, R2 are the same as defined above.

The reaction processes are exemplified in the following stepwise reaction.

The compound of formula (VII) is, for example, subjected to a condensation reaction with trimethylacetyl chloride to yield the compound of formula (VI) under pyridine condition.

Subsequently, the compound of formula (VI) is allowed to react with, for example, N-chlorosuccinimide in an organic solvent such as acetonitrile at room temperature to yield chlorinated compound of formula (V), followed by stirring under sulfuric acid and nitric acid at 65 to 75° C. to obtain the compound of formula (IV) containing a nitro group.

Next, the compound of formula (IV) obtained above is subjected to react with a compound having an acrylamide group, e.g., N-(3-hydroxyphenyl)acrylamide, under a solvent such as N,N-dimethylformamide and an inorganic base such as cesium carbonate at 30 to 40° C. to yield the compound of formula (III) containing an acryl amide group.

The nitro group of the compound of formula (III) may be converted to an amino group by subjecting the compound to an iron-mediated reduction reaction or a hydrogenation reaction using palladium/carbon as a catalyst to obtain the aniline compound of formula (II), which is subjected to a further reaction with various Z-substituted aldehydes in a solvent such as dimethylformamide under the presence of ferric chloride at 115 to 125° C. to obtain the target compound of formula (I).

The compound of formula (I) of the present invention may also form a pharmaceutically acceptable organic or inorganic acid addition salts. Examples of such salts are acid addition salts formed by acids such as hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid and toluenesulfonic acid.

Specifically, the pharmaceutically acceptable salt in accordance with the present invention can be prepared by dissolving the compound of formula (I) in a water miscible organic solvent, e.g., acetone, methanol, ethanol or acetonitrile, followed by addition of organic or inorganic acid, and filtering the precipitated crystal. Also, it may be prepared by removing under reduced pressure a solvent or an excessive amount of acid from a reaction mixture with an added acid, followed by drying the residue, or conducting eduction using a different organic solvent, followed by filtering the precipitated salt.

The compound of formula (I) in accordance with the present invention or a pharmaceutically acceptable salt thereof may be in the form of solvates or hydrates, and such compounds are also included within the scope of the present invention.

The compound of formula (I) in accordance with the present invention or a pharmaceutically acceptable salt thereof can selectively and effectively inhibit a protein kinase. In one embodiment, such compound can selectively and effectively inhibit kinases that are mostly expressed in aberrantly activated lymphocytes (T-lymphocytes and/or B-lymphocytes) including Janus kinase 3 (JAK3), Bruton's tyrosine kinase (BTK), IL-2 inducing T-cell kinase (ITK), resting lymphocyte kinase (RLK) and bone marrow tyrosine kinase (BMX), and thus, may be useful for prevention or treatment of diseases that are mediated by aberrantly activated T-lymphocytes, B-lymphocytes or both such as inflammatory diseases, autoimmune diseases, proliferative diseases or hyperproliferative diseases, immunologically mediated diseases, cancers or tumors. Therefore, the present invention provides a pharmaceutical composition comprising the compound of formula (I) or a pharmaceutically acceptable salt as an active ingredient for prevention or treatment of inflammatory diseases, autoimmune diseases, proliferative diseases or hyperproliferative diseases, immunologically mediated diseases, cancers or tumors.

Examples of said inflammatory diseases, autoimmune diseases, proliferative diseases or hyperproliferative diseases or immunologically mediated diseases may be selected from the group consisting of arthritis, rheumatoid arthritis, spondyloarthropathy, gouty arthritis, osteoarthritis, juvenile arthritis, other arthritic conditions, lupus, systemic lupus erythematosus (SLE), skin-related diseases, psoriasis, eczema, dermatitis, atopic dermatitis, pain, pulmonary disorder, lung inflammation, adult respiratory distress syndrome (ARDS), pulmonary sarcoidosis, chronic pulmonary inflammatory disease, chronic obstructive pulmonary disease (COPD), cardiovascular disease, artherosclerosis, myocardial infarction, congestive heart failure, cardiac reperfusion injury, inflammatory bowel disease, Crohn's disease, ulcerative colitis, irritable bowel syndrome, asthma, Sjogren's syndrome, autoimmune thyroid disease, urticaria, multiple sclerosis, scleroderma, allograft rejection, xenotransplantation, idiopathic thrombocytopenic purpura (ITP), Parkinson's disease, Alzheimer's disease, diabetic associated disease, inflammation, pelvic inflammatory disease, allergic rhinitis, allergic bronchitis, allergic sinusitis, leukemia, lymphoma, B-cell lymphoma, T-cell lymphoma, myeloma, acute lymphoid leukemia (ALL), chronic lymphoid leukemia (CLL), acute myeloid leukemia (AML), chronic myeloid leukemia (CML), hairy cell leukemia, Hodgkin's disease, non-Hodgkin's lymphoma, multiple myeloma, myelodysplastic syndrome (MDS), myeloproliferative neoplasms (MPN), diffuse large B-cell lymphoma and follicular lymphoma, but not limited thereto.

Further, examples of said cancer and tumor may be selected from the group consisting of liver cancer, hepatocellular carcinoma, thyroid cancer, colon cancer, testicular cancer, bone cancer, oral cancer, basal cell carcinoma, ovarian cancer, brain tumor, gallbladder carcinoma, biliary tract cancer, head and neck cancer, vesical carcinoma, tongue cancer, esophageal cancer, glioma, glioblastoma, renal cancer, malignant melanoma, gastric cancer, breast cancer, sarcoma, pharynx carcinoma, uterine cancer, cervical cancer, prostate cancer, rectal cancer, pancreatic cancer, lung cancer, skin cancer and other solid tumor, but not limited thereto.

The compound of formula (I) of the present invention or a pharmaceutically acceptable salt thereof may be used in combination with other drugs to enhance efficacy in treatment of inflammatory diseases, autoimmune diseases, proliferative diseases or hyperproliferative diseases, or immunologically mediated diseases.

Examples of the drug which may be used in combination with the inventive compound or a pharmaceutically acceptable salt thereof for treatment of inflammatory diseases, autoimmune diseases, proliferative diseases or hyperproliferative diseases, or immunologically mediated diseases are one or more of drugs selected from the group consisting of steroids (prednisone, prednisolone, methylprednisolone, cortisone, hydroxycortisone, betamethasone, dexamethasone, etc.), methotrexate, lefluonomide, anti-TNF-α agents (etanercept, infliximab, adalimumab, etc.), calcineurin inhibitors (tacrolimus, pimecrolimus, etc.) and antihistamines (diphenhydramine, hydroxyzine, loratadine, ebastine, ketotifen, cetirizine, levocetirizine, fexofenadine, etc.), but not limited thereto.

Examples of the drug which may be used in combination with the inventive compound or a pharmaceutically acceptable salt thereof for treatment of cancers or tumors include one or more selected from the group consisting cell signaling inhibitors (glivec, iressa, tarceva, etc.), mitotic inhibitor (vincristine, vinblastine, etc.), alkylating agents (cyclophosphamide, thiotepa, busulfan, etc.), antimetabolites (tagafur, methotrexate, gemcitabine, etc.), intercalating agents (proflavin, ethidium bromide, actinomycin D, etc.), topoisomerase inhibitors (irinotecan, topotecan, amsacrine, etoposide, teniposide, etc.), immunotherapeutic agents (interferon α, β, γ, interleukin, etc.) and antihormonal agents (tamoxifen, leuprorelin, anastrozole, etc.), but not limited thereto.

A proposed daily dose of the compound of formula (I) or a pharmaceutically acceptable salt thereof for administration to a human (of approximately 70 kg body weight) may be in the range of 0.1 mg/day to 2,000 mg/day, preferably 1 mg/day to 1,000 mg/day, 1 to 4 times daily or on/off schedule by oral or parenteral administration. The inventive compound may be administered in a single dose or in divided doses per day. It is understood that the daily dose should be determined in light of various relevant factors including the condition, age, body weight and sex of the subject to be treated, administration route, and disease severity, and therefore the dosage suggested above should not be construed to limit the scope of the present invention in any way.

The pharmaceutical composition of the present invention may typically comprise pharmaceutically acceptable additives, carriers or excipients. The pharmaceutical composition of the present invention may be formulated in accordance with conventional methods, and may be prepared in the form of oral formulations such as tablets, pills, powders, capsules, syrups, emulsions, microemulsions and others, or parenteral formulations such as intramuscular, intravenous or subcutaneous administrations.

For oral formulations, carriers or additives such as cellulose, calcium silicate, corn starch, lactose, sucrose, dextrose, calcium phosphate, stearic acid, magnesium stearate, calcium stearate, gelatin, talc, surfactants, suspending agents, emulsifiers, diluents, and others may be used. For injectable formulations, carriers or additives such as water, saline, glucose solution, glucose solution analogs, alcohols, glycols, ethers (e.g., polyethylene glycol 400), oils, fatty acids, fatty acid esters, glycerides, surfactants, suspending agents, emulsifiers, and others may be used.

Also, the present invention provides a method for preventing or treating inflammatory disease, autoimmune disease, proliferative disease or hyperproliferative disease, immunologically mediated disease in an mammal, comprising the step of administering to the mammal an effective amount of the compound of formula (I) or a pharmaceutically acceptable salt thereof.

The compound of formula (I) of the present invention may be used for the study of biological and pathological phenomena of a kinase, the study of intracellular signaling pathway mediated by a kinase as well as comparative evaluation with new kinase inhibitors.

### EXAMPLES

The following Examples are provided to illustrate preferred embodiments of the present invention, and are not intended to limit the scope of the present invention.

### Example 1

### Preparation of N-(3-(6-chloro-2-(4-(4-methylpiperazin-1-yl)phenyl)-3H-imidazo[4,5-b]pyridin-7-yloxy)phenyl)acrylamide

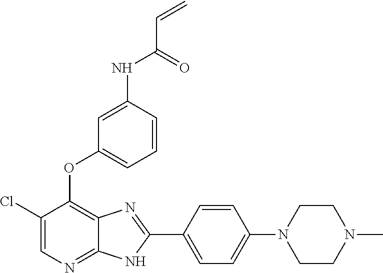 [SMILES: C=CC(=O)Nc1cccc(Oc2c(Cl)cnc3[nH]c(-c4ccc(N5CCN(C)CC5)cc4)nc23)c1]

### Step 1) Preparation of 4-chloro-2-trimethylacetamidopyridine

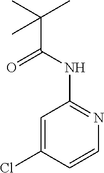

Pyridine (40 mL) was added to 2-amino-4-chloropyridin (10.0 g, 0.077 mol) and trimethylacetyl chloride (10.4 mL, 0.116 mol), followed by stirring for 5 hours at room temperature. The reaction solution was distilled under reduced pressure, added with ethylacetate, and washed with an aqueous solution of saturated sodium bicarbonate. The organic layer was separated, dried over anhydrous sodium sulfate, filtered and distilled under reduced pressure. The resulting solid was dried under reduced pressure to obtain the title compound (11.5 g, yield: 70%).

¹H-NMR (300 MHz, DMSO-d₆) δ 1.24 (s, 9H), 7.25 (dd, 1H), 8.17(d, 1H), 8.34 (d, 1H), 10.09 (s, 1H)

### Step 2) Preparation of 4,5-dichloro-2-trimethylacetamidopyridine

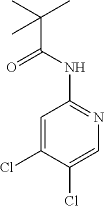

Acetonitrile (200 mL) was added to the compound obtained in Step 1 (11.0 g, 0.051 mol) and N-chlorosuccinimide (34.0 g, 0.255 mol), followed by stirring for 5 hours at 100° C. The reaction solution was distilled under reduced pressure, added with ethylacetate, and washed with an aqueous solution of saturated sodium bicarbonate. The organic layer was separated, dried over anhydrous sodium sulfate, filtered and distilled under reduced pressure. The resulting solid was dried under reduced pressure to obtain the title compound (6.3 g, yield: 50%).

¹H-NMR (300 MHz, DMSO-d₆) δ 1.21 (s, 9H), 8.33 (s, 1H), 8.54 (s, 1H), 10.30 (s, 1H)

### Step 3) Preparation of 2-amino-4,5-dichloro-3-nitropyridine

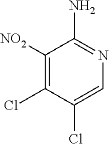 [SMILES: Nc1ncc(Cl)c(Cl)c1[N+](=O)[O-]]

The compound obtained in Step 2 (6.3 g, 0.025 mol) was dissolved in concentrated sulfuric acid (100 mL) at 100° C., and 60˜62% nitric acid (2.4 mL) was added dropwise, followed by stirring for 1 hour at 65˜70° C. The reaction solution was cooled to 0° C., and the pH value was adjusted to 7.0 using a 2 N aqueous solution of sodium chloride to obtain a solid, which was then subjected to stirring for 2 hours. The solid obtained was filtered under reduced pressure, washed with distilled water and dried to obtain the title compound (2.08 g, yield: 40%).

¹H-NMR (300 MHz, DMSO-d₆) δ 7.35 (s, 2H), 8.37 (s, 1H)

### Step 4) Preparation of N-(3-hydroxyphenyl)acrylamide

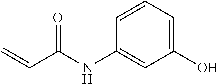

Tetrahydrofuran (100 mL) and water (30 mL) was added to 3-aminophenyl (5.0 g, 0.045 mol), sodium hydrogen carbonate (5.6 g, 0.067 mol) was added thereto, and acroyl chloride (3.6 mL, 0.045 mol) was added dropwise. The reaction solution was stirred for 3.5 hours at room temperature, added with dichloromethane, and washed with an aqueous solution of ammonium chloride. The organic layer was separated, dried over sodium sulfate, filtered and distilled under reduced pressure. The resulting residue was distilled with dichloromethane and ether to obtain the title compound (2.9 g, yield: 40%).

¹H-NMR (300 MHz, DMSO-d₆) δ 5.73 (dd, 1H), 6.24 (dd, 1H), 6.42 (m, 2H), 7.04 (m, 2H), 7.24(s, 1H), 9.39 (s, 1H), 9.97 (s, 1H)

### Step 5) Preparation of N-(3-((2-amino-5-chloro-3-nitropyridin-4-yl)oxy)phenyl)acrylamide

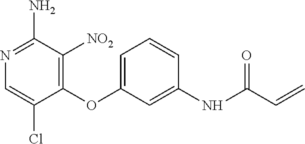 [SMILES: C=CC(=O)Nc1cccc(Oc2c(Cl)cnc(N)c2[N+](=O)[O-])c1]

N,N-dimethylformamide (40 mL) was added to the compound obtained in Step 3 (2.0 g, 0.010 mol) and cesium carbonate (4.7 g, 0.001 mol), followed by stirring for 30 minutes at room temperature. The compound obtained in Step 4 (2.28 g, 0.001 mol) was slowly added thereto, followed by stirring for 5 hours at 35° C. Ethylacetate was added to the reaction solution, and washed with water several times. The organic layer was separated, dried over anhydrous sodium sulfate, filtered and distilled under reduced pressure. The resulting residue was separated by column chromatography (chloroform:methanol=20:1 (v:v)) to obtain the title compound (2.0 g, yield: 65%).

¹H-NMR (300 MHz, DMSO-d₆) δ 5.72 (dd, 1H), 6.35 (dd, 1H), 6.41 (m, 2H), 6.69 (d, 1H), 7.03 (m, 2H), 7.32 (s, 1H), 7.50 (s, 1H), 8.46(s, 1H), 10.25 (s, 1H)

### Step 6) Preparation of N-(3-((2,3-diamino-5-chloropyridin-4-yl)oxy)phenyl)acrylamide

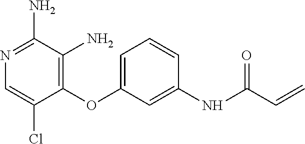 [SMILES: C=CC(=O)Nc1cccc(Oc2c(Cl)cnc(N)c2N)c1]

Iron (3.57 g, 0.063 mol) and 12 N aqueous solution of hydrochloric acid (0.64 mL) were diluted in an aqueous solution of 50% ethanol (60 mL), followed by stirring for 1 hour at 100° C. The compound obtained in Step 5 (2 g, 0.006 mol) was added to the reaction solution, followed by stirring for 1 hour at 100° C. The reaction solution was filtered through a Cellite filter under reduced pressure and distilled under reduced pressure. Dichloromethane was added to the obtained residue, followed by washing with an aqueous solution of saturated sodium bicarbonate. The organic layer was separated, dried over anhydrous sodium sulfate, filtered and distilled under reduced pressure. The resulting residue was separated by column chromatography (dichloromethane:methanol=10:1 (v:v)) to obtain the title compound (1.25 g, yield: 70%).

¹H-NMR (300 MHz, DMSO-d₆) δ 4.86 (s, 2H), 5.73 (dd, 1H), 5.92 (s, 2H), 6.32 (m, 2H), 6.59 (d, 1H), 7.14 (s, 1H), 7.26 (t, 1H), 7.45 (s, 2H), 10.35 (s, 1H)

### Step 7) Preparation of 4-(4-(methylpiperazin-1-yl)benzaldehyde

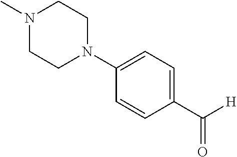

Water (30 mL) was added to 4-fluorobenzaldehyde (3.0 g, 0.024 mol) and sodium carbonate (3.83 g, 0.036 mol). 1-methylpiperazin (4.11 g, 0.041 mol) was slowly added dropwise thereto, followed by stirring for 12 hours at 0° C. Dichloromethane was added to the reaction solution, and washed with an aqueous solution of saturated sodium bicarbonate. The organic layer was separated, dried over anhydrous sodium sulfate, filtered and distilled under reduced pressure, and the resulting residue was crystallized with n-hexane to obtain the title compound (2.9 g, yield:40%).

¹H-NMR (300 MHz, DMSO-d₆) δ 2.21 (s, 3H), 2.42 (m, 4H), 3.35 (m, 4H), 7.02 (d, 2H), 7.69 (d, 2H), 9.71 (s, 1H)

### Step 8) Preparation of N-(3-(6-chloro-2-(4-(4-methylpiperazin-1-yl)phenyl)-3H-imidazo[4,5-b]pyridin-7-yloxy)phenyl)acrylamide

N,N-dimethylformamide (30 mL) was added to N-(3-((2,3-diamino-5-chloropyridin-4-yl)oxy)phenyl)acrylamide obtained in Step 6 (1.25 g, 0.004 mol) and 4-(4-methylpiperazin-1-yl)benzaldehyde obtained in Step 7 (0.83 g, 0.004 mol). Ferric chloride (0.033 g, 0.123 mmol) was added to the mixture, followed by stirring for 6 hours at 120° C. Dichloromethane was added to the reaction solution, and washed with water several times. The organic layer was separated, dried over anhydrous sodium sulfate, filtered and distilled under reduced pressure. The resulting residue was separated by column chromatography (chloroform:methanol=20:1 (v:v)) to obtain the title compound (0.8 g, yield: 40%).

¹H-NMR (300 MHz, DMSO-d₆) δ 2.18 (s, 2H), 2.38 (m, 4H), 3.33 (m, 4H), 5.73 (dd, 1H), 6.32 (m, 2H), 6.55 (d, 1H), 7.05 (d, 2H), 7.14 (s, 1H), 7.22 (t, 1H), 7.41 (s, 2H), 7.69 (d, 2H), 10.35 (s, 1H), 13.45 (s, 1H)

MS (ESI³⁰): m/z=489.2 [M+H]⁺

### Example 2

### Preparation of N-(3-(6-chloro-2-(4-(4-hydroxypiperidin-1-yl)phenyl)-3H-imidazo[4,5-b]pyridin-7-yloxy)phenyl)acrylamide

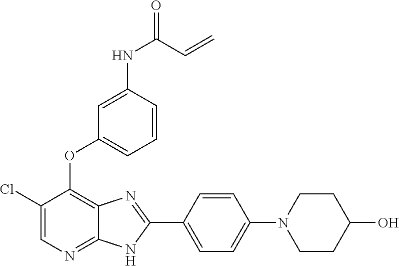 [SMILES: C=CC(=O)Nc1cccc(Oc2c(Cl)cnc3[nH]c(-c4ccc(N5CCC(O)CC5)cc4)nc23)c1]

### Step 1) Preparation of 4-(4-hydroxypiperidin-1-yl)benzaldehyde

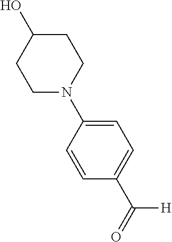 [SMILES: O=Cc1ccc(N2CCC(O)CC2)cc1]

Dimethylformamide (15 mL) was added to 4-fluorobenzaldehyde (1.5 g, 0.012 mol) and sodium carbonate (2.5 g, 0.018 mol). Piperidin-4-ol (1.47 g, 0.015 mol) was slowly added dropwise thereto, followed by stirring for 8 hours at 70 to 80° C. Dichloromethane was added to the reaction solution, and the mixture was washed with an aqueous solution of saturated sodium bicarbonate. The organic layer was separated, dried over anhydrous sodium sulfate, filtered and distilled under reduced pressure. The resulting residue was crystallized with n-hexane to obtain the title compound (1.5 g, yield: 52%).

¹H-NMR (300 MHz, DMSO-d₆) δ 1.40 (m, 2H), 1.79 (m, 2H), 3.10 (m, 2H), 3.75 (m, 3H), 4.77 (d, 1H), 7.02 (d, 2H), 7.68 (d, 2H), 9.67 (s, 1H)

### Step 2) Preparation of N-(3-(6-chloro-2-(4-(4-hydroxypiperidin-1-yl)phenyl)-3H-imidazo[4,5-b]pyridin-7-yloxy)phenyl)acrylamide

The procedure of Step 8 of Example 1 was repeated, except for using 4-(4-hydroxypiperidin-1-yl)benzaldehyde obtained in Step 1 above and N-(3-((2,3-diamino-5-chloropyridin-4-yl)oxy)phenyl)acrylamide obtained in Step 6 of Example 1 to obtain the compound of Example 2.

¹H-NMR (300 MHz, DMSO-d₆) δ 1.41 (m, 2H), 1.79 (m, 2H), 2.99 (m, 2H), 3.69 (m, 3H), 4.71 (d, 1H), 5.72 (d, 1H), 6.20 (m, 1H), 6.34 (m, 1H), 6.74 (d, 1H), 7.02 (d, 2H), 7.29 (m, 2H), 7.41 (d, 1H), 7.95 (br, 1H), 8.37 (s, 1H), 10.15 (s, 1H)

MS (ESI⁺): m/z=490.2 [M+H]⁺

### Examples 3 to 19

Various aldehyde derivatives expressed as W—COH (W is as defined above) were prepared by using the methods same as or similar to those for preparing 4-(4-methylpiperazin-1-yl)benzaldehyde obtained in Step 7 of Example 1 or 4-(4-hydroxypiperidin-1-yl)benzaldehyde obtained in Step 1 of Example 2, and then the procedure of Step 8 of Example 1 was repeated to obtain the compounds of Examples 3 to 19 as shown in Table 1.

### Example 20

### Preparation of N-(3-(2-(4-(4-methylpiperazin-1-yl)phenyl)-3H-imidazo[4,5-b]pyridin-7-yloxy)phenyl)acrylamide

The procedure of Example 1 was repeated, except for using 2-aminopyridine instead of 2-amino-4-chloropyridine used in Step 1 of Example 1, to obtain the compound of Example 20 as shown in Table 1.

TABLE 1

| Ex. | Structure | Analysis Data |
|---|---|---|
| 3 | 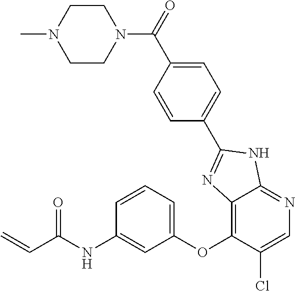 [SMILES: C=CC(=O)Nc1cccc(Oc2c(Cl)cnc3[nH]c(-c4ccc(C(=O)N5CCN(C)CC5)cc4)nc23)c1] | ¹H-NMR (300 MHz, DMSO-d₆) d 10.20 (s, 1H), 8.53 (s, 1H), 8.20 (m, 2H), 7.58 (d, 2H), 7.34 (m, 3H), 6.75 (d, 1H), 6.32 (dd, 1H), 6.20 (d, 1H), 5.75 (d, 1H), 3.55 (m, 4H), 2.70 (m, 4H), 2.40 (s, 3H); MS (ESI⁺): m/z = 517.2 [M + H]⁺ |
| 4 | 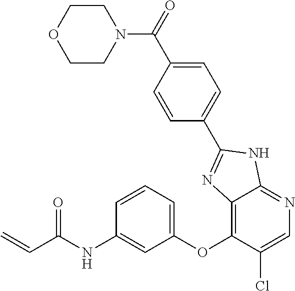 [SMILES: C=CC(=O)Nc1cccc(Oc2c(Cl)cnc3[nH]c(-c4ccc(C(=O)N5CCOCC5)cc4)nc23)c1] | ¹H-NMR (300 MHz, DMSO-d₆) d 10.17 (s, 1H), 8.52 (s, 1H), 8.20 (m, 2H), 7.56 (d, 2H), 7.34 (m, 3H), 6.78 (d, 1H), 6.36 (dd, 1H), 6.20 (d, 1H), 5.73 (d, 1H), 3.55 (s, 8H); MS (ESI⁺): m/z = 504.1 [M + H]⁺ |
| 5 | 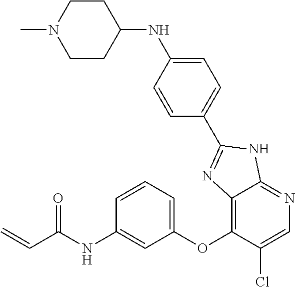 [SMILES: C=CC(=O)Nc1cccc(Oc2c(Cl)cnc3[nH]c(-c4ccc(NC5CCN(C)CC5)cc4)nc23)c1] | ¹H-NMR (300 MHz, DMSO-d₆) d 10.15 (s, 1H), 8.48 (s, 1H), 7.85 (m, 2H), 7.42 (d, 1H), 7.27 (m, 2H), 6.73 (dd, 1H), 6.54 (d, 2H), 6.37 (dd, 1H), 6.26 (m, 2H), 5.72 (dd, 1H), 2.73 (m, 2H), 2.12 (s, 3H), 2.02 (t, 2H), 1.88 (d, 2H), 1.42 (m, 2H); MS (ESI⁺): m/z = 503.2 [M + H]⁺ |
| 6 | 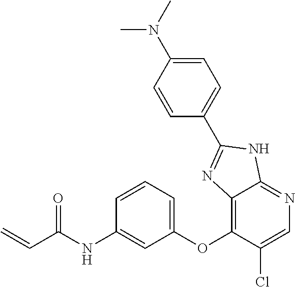 [SMILES: C=CC(=O)Nc1cccc(Oc2c(Cl)cnc3[nH]c(-c4ccc(N(C)C)cc4)nc23)c1] | ¹H-NMR (300 MHz, DMSO-d₆) d 10.18 (s, 1H), 8.51 (s, 1H), 8.16 (m, 2H), 7.58 (d, 2H), 7.37 (m, 3H), 6.75 (dd, 1H), 6.35 (dd, 1H), 6.20 (dd, 1H), 5.70 (d, 1H), 3.04 (s, 6H); MS (ESI⁺): m/z = 434.1 [M + H]⁺ |
| 7 | 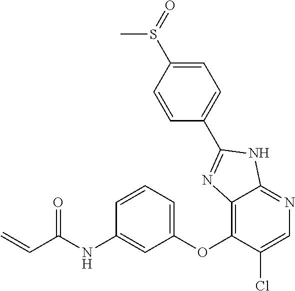 [SMILES: C=CC(=O)Nc1cccc(Oc2c(Cl)cnc3[nH]c(-c4ccc(S(C)=O)cc4)nc23)c1] | ¹H-NMR (300 MHz, DMSO-d₆) d 10.15 (s, 1H), 8.52 (s, 2H), 8.19 (m, 2H), 7.54 (d, 2H), 7.40 (m, 3H), 6.77 (dd, 1H), 6.37 (dd, 1H), 6.22 (dd, 1H), 5.75 (d, 1H), 2.66 (s, 3H); MS (ESI⁺): m/z = 453.1 [M + H]⁺ |
| 8 | 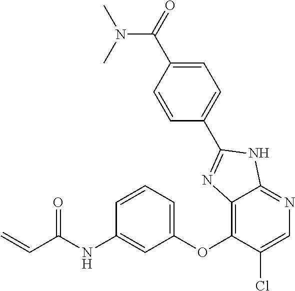 [SMILES: C=CC(=O)Nc1cccc(Oc2c(Cl)cnc3[nH]c(-c4ccc(C(=O)N(C)C)cc4)nc23)c1] | ¹H-NMR (300 MHz, DMSO-d₆) d 10.17 (s, 1H), 8.51 (s, 1H), 8.16 (m, 2H), 7.54 (d, 2H), 7.37 (m, 3H), 6.77 (dd, 1H), 6.35 (dd, 1H), 6.20 (dd, 1H), 5.70 (d, 1H), 2.99 (s, 3H), 2.91 (s, 3H); MS (ESI⁺): m/z = 462.1 [M + H]⁺ |
| 9 | 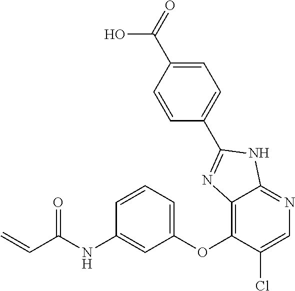 [SMILES: C=CC(=O)Nc1cccc(Oc2c(Cl)cnc3[nH]c(-c4ccc(C(=O)O)cc4)nc23)c1] | ¹H-NMR (300 MHz, DMSO-d₆) d 10.20 (s, 1H), 8.40 (s, 1H), 8.13 (d, 2H), 7.97 (d, 2H), 7.42 (d, 1H), 7.29 (m, 2H), 6.74 (dd, 1H), 6.38 (dd, 1H), 6.22 (dd, 1H), 5.69 (dd, 1H); MS (ESI⁺): m/z = 435.8 [M + H]⁺ |
| 10 | 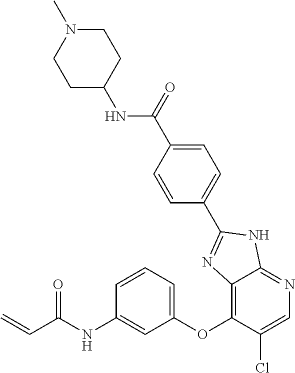 [SMILES: C=CC(=O)Nc1cccc(Oc2c(Cl)cnc3[nH]c(-c4ccc(C(=O)NC5CCN(C)CC5)cc4)nc23)c1] | ¹H-NMR (300 MHz, DMSO-d₆) d 10.18 (s, 1H), 8.44 (s, 1H), 8.39 (d, 1H), 8.21 (d, 1H), 7.95 (d, 1H), 7.35 (m, 2H), 6.75 (m, 1H), 6.29 (m, 1H), 6.22 (m, 1H), 5.73 (d, 1H), 2.75 (m, 3H), 2.16 (s, 3H), 1.95 (m, 2H), 1.74 (m, 2H), 1.59 (m, 2H); MS (ESI⁺): m/z = [M + H]⁺ |
| 11 | 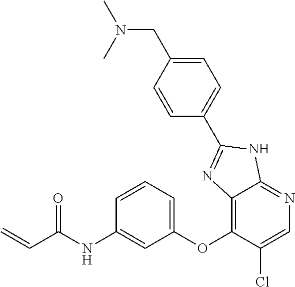 [SMILES: C=CC(=O)Nc1cccc(Oc2c(Cl)cnc3[nH]c(-c4ccc(CN(C)C)cc4)nc23)c1] | ¹H-NMR (300 MHz, DMSO-d₆) d 10.19 (s, 1H), 8.49 (s, 1H), 8.10 (d, 2H), 7.35 (m, 5H), 6.76 (d, 1H), 6.36 (dd, 1H), 6.19 (dd, 1H), 5.72 (dd, 1H), 3.45 (s, 2H), 2.15 (s, 6H); MS (ESI⁺): m/z = 448.1 [M + H]⁺ |
| 12 | 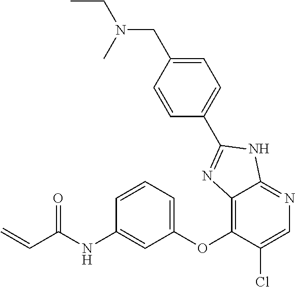 [SMILES: C=CC(=O)Nc1cccc(Oc2c(Cl)cnc3[nH]c(-c4ccc(CN(C)CC)cc4)nc23)c1] | ¹H-NMR (300 MHz, DMSO-d₆) d 10.17 (s, 1H), 8.48 (s, 1H), 8.09 (m, 2H), 7.43 (m, 3H), 7.30 (m, 2H), 6.76 (d, 1H), 6.34 (m, 1H), 6.20 (dd, 1H), 6.72 (dd, 1H), 3.50 (s, 2H), 2.39 (dd, 2H), 2.10 (s, 3H), 1.02 (t, 3H); MS (ESI⁺): m/z = 462.0 [M + H]⁺ |
| 13 | 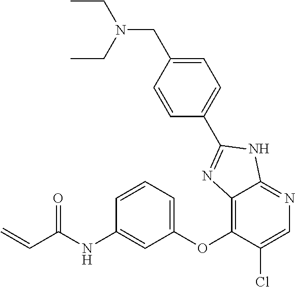 [SMILES: C=CC(=O)Nc1cccc(Oc2c(Cl)cnc3[nH]c(-c4ccc(CN(CC)CC)cc4)nc23)c1] | ¹H-NMR (300 MHz, DMSO-d₆) d 10.18 (s, 1H), 8.48 (s, 1H), 8.06 (d, 2H), 7.33 (m, 5H), 6.76 (d, 1H), 6.35 (dd, 1H), 6.20 (dd, 1H), 5.72 (dd, 1H), 3.59 (d, 2H), 2.49 (m, 4H), 0.98 (t, 6H); MS (ESI⁺): m/z = 476.1 [M + H]⁺ |
| 14 | 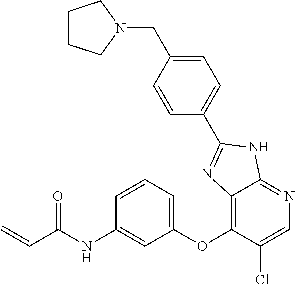 [SMILES: C=CC(=O)Nc1cccc(Oc2c(Cl)cnc3[nH]c(-c4ccc(CN5CCCC5)cc4)nc23)c1] | ¹H-NMR (300 MHz, DMSO-d₆) d 10.20 (s, 1H), 8.48 (s, 1H), 8.09 (m, 2H), 7.47-7.27 (m, 5H), 6.75 (dd, 1H), 6.34 (m, 1H), 6.19 (dd, 1H), 5.72 (dd, 1H), 3.65 (s, 2H), 2.46 (m, 4H), 1.69 (m, 4H); MS (ESI⁺): m/z = 474.0 [M + H]⁺ |
| 15 | 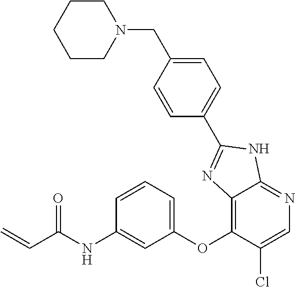 [SMILES: C=CC(=O)Nc1cccc(Oc2c(Cl)cnc3[nH]c(-c4ccc(CN5CCCCC5)cc4)nc23)c1] | ¹H-NMR (300 MHz, DMSO-d₆) d 10.19 (s, 1H), 8.48 (s, 1H), 8.07 (d, 1H), 7.33 (m, 5H), 6.76 (d, 1H), 6.35 (dd, 1H), 6.22 (dd, 1H), 5.72 (dd, 1H), 2.35 (m, 4H), 1.45 (m, 6H); MS (ESI⁺): m/z = 488.1 [M + H]⁺ |
| 16 | 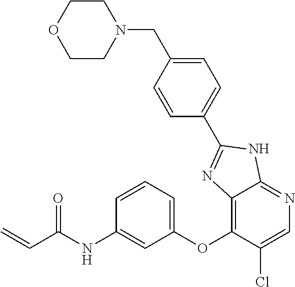 [SMILES: C=CC(=O)Nc1cccc(Oc2c(Cl)cnc3[nH]c(-c4ccc(CN5CCOCC5)cc4)nc23)c1] | ¹H-NMR (300 MHz, DMSO-d₆) d 10.10 (s, 1H), 8.39 (s, 1H), 8.01 (m, 2H), 7.33-7.19 (m, 5H), 6.67 (d, 1H), 6.25 (m, 1H), 6.11 (d, 1H), 5.63 (d, 1H), 3.48 (m, 4H), 3.42 (s, 2H), 2.26 (m, 4H); MS (ESI⁺): m/z = 490.1 [M + H]⁺ |
| 17 | 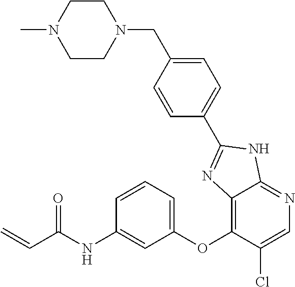 [SMILES: C=CC(=O)Nc1cccc(Oc2c(Cl)cnc3[nH]c(-c4ccc(CN5CCN(C)CC5)cc4)nc23)c1] | ¹H-NMR (300 MHz, DMSO-d₆) d 10.20 (s, 1H), 8.49 (s, 1H), 8.10 (m, 2H), 7.36 (m, 5H), 6.75 (m, 1H), 6.27 (dd, 1H), 6.18 (dd, 1H), 5.72 (dd, 1H), 3.57 (s, 2H), 2.49 (m, 8H), 2.30 (s, 3H); MS (ESI⁺): m/z = 503.1 [M + H]⁺ |
| 18 | 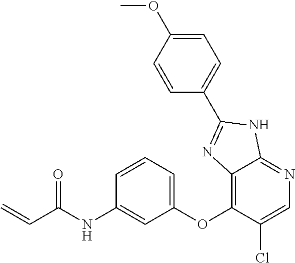 [SMILES: C=CC(=O)Nc1cccc(Oc2c(Cl)cnc3[nH]c(-c4ccc(OC)cc4)nc23)c1] | ¹H-NMR (300 MHz, DMSO-d₆) d 10.16 (s, 1H), 8.42 (s, 1H), 8.09 (m, 2H), 7.32 (m, 3H), 7.08 (m, 2H), 6.76 (dd, 1H), 6.32 (dd, 1H), 6.19 (dd, 1H), 5.72 (dd, 1H), 3.82 (s, 3H); MS (ESI⁺): m/z = 421.1 [M + H]⁺ |
| 19 | 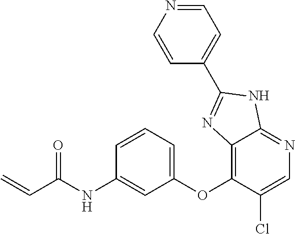 [SMILES: C=CC(=O)Nc1cccc(Oc2c(Cl)cnc3[nH]c(-c4ccncc4)nc23)c1] | ¹H-NMR (300 MHz, DMSO-d₆) d 10.19 (s, 1H), 8.75 (d, 2H), 8.58 (d, 1H), 8.05 (br, 2H), 7.39 (m, 2H), 7.31 (t, 1H), 6.78 (d, 1H), 6.35 (m, 1H), 8.20 (dd, 1H), 5.73 (dd, 1H); MS (ESI⁺): m/z = 392.1 [M + H]⁺ |
| 20 | 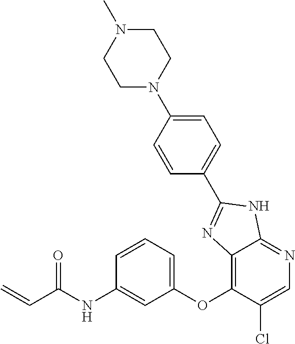 | ¹H-NMR (300 MHz, DMSO-d₆) d 8.26 (d, 1H), 8.05 (d, 2H), 7.72 (s, 1H), 7.44 (m, 3H), 7.10 (d, 2H), 7.00 (d, 1H), 6.62 (d, 1H), 6.40 (m, 2H), 5.77 (dd, 1H), 3.37 (m, 4H), 2.63 (m, 4H), 2.36 (s, 3H); MS (ESI⁺): m/z = 455.2 [M + H]⁺ |

### Formulation Example 1

### Preparation of Tablet

According to a conventional method, the following composition formula given in Table 2 below was used to prepare a single tablet for oral administration by using each of the compounds prepared in Examples 1 to 20 as an active ingredient.

TABLE 2

| Composition | Amount |
|---|---|
| Active ingredient | 100 mg |
| Corn starch | 80 mg |
| Lactose | 80 mg |
| Magnesium stearate | 5 mg |

### Formulation Example 2

### Preparation of Capsule

According to a conventional method, the following composition formula given in Table 3 below was used to prepare a hard gelatin capsule for oral administration by using each of the compounds prepared in Examples 1 to 20 as an active ingredient.

TABLE 3

| Composition | Amount |
|---|---|
| Active ingredient | 100 mg |
| Corn starch | 40 mg |
| Lactose | 80 mg |
| Crystalline cellulose | 80 mg |
| Magnesium stearate | 5 mg |

### Formulation Example 3

### Preparation of Injectable Pharmaceutical Formulation

According to a conventional method, the following composition formula given in Table 4 below was used to prepare an injectable pharmaceutical formulation by using each of the compounds prepared in Examples 1 to 20 as an active ingredient. However, pH value was not controlled when the salt of the compound of formula (I) was used as an active ingredient.

TABLE 4

| Composition | Amount |
|---|---|
| Active ingredient | 20 mg |
| 5% glucose solution | 10 mL |
| HCl (1N) | Suitable for keeping pH of 4 |

### Formulation Example 4

### Preparation of Injectable Pharmaceutical Formulation

According to a conventional method, the following composition formula given in Table 5 below was used to prepare an injectable pharmaceutical formulation by using each of the compounds prepared in Examples 1 to 20 as an active ingredient.

TABLE 5

| Composition | Amount |
|---|---|
| Active ingredient | 20 mg |
| Polyethylene glycol 400 | 2 mL |
| Sterile water | 8 mL |

### Test Example 1

### Evaluation of JAK3 and BTK Inhibitory Activity

The compounds prepared in Examples 1 to 20 were tested for JAK3 and BTK inhibitory activity. Kinase inhibitory activity was measured by using Z-Lyte Kinase Assay Kit (Invitrogen), and JAK3 and BTK enzymes were purchased from Invitrogen (PV3855, PV3190).

Specifically, the compounds of Examples 1 to 20 were diluted with a 4% aqueous solution of DMSO to obtain solutions with concentrations in the range of 1˜0.0001 μM. Each kinase was diluted to 1˜10 ng/assay, and ATP was diluted to form a kinase buffer (50 mM HEPES, pH 7.4; 10 mM MgCl₂; 1 mM EGTA; 0.01% BRIJ-35) by calculating an approximate Kd value. The assays were performed in 384-well polystyrene flat-bottomed plates. 5 μL of the diluted solution of the compound, peptide substrate having a suitable concentration, 10 μL of mixed kinase solution and 5 μL of ATP solution having a concentration of 5 to 300 μM were added to the sample, and allowed to react in a mixer for 60 minutes at room temperature. After 60 minutes, 10 μL of fluorescent labeling reagents was added to each mixture so as to allow fluorescent labeling of peptide substrates, followed by adding a finishing solution to complete the reaction. The fluorescence level was measured using a Molecular Device with an excitation filter at 400 nm, and an emission filter at 520 nm. The kinase inhibitory activities of the compounds were calculated in phosphorylation rates between 0˜100% against the control group (staurosporine or each of kinase inhibitor) with reference to the protocol of the kit, and percentage inhibition was calculated and plotted against concentration (x-axis) to calculate 50% inhibitory concentration (IC₅₀). The IC₅₀ values were obtained by using Microsoft Excel, and the results are shown in Table 5, wherein A: IC₅₀≦50 nM, B: IC₅₀=50˜100 nM, C: IC₅₀=100˜1,000 nM, and D: IC₅₀≧1,000 nM.

TABLE 6
IC₅₀

| Example | JAK3 | BTK |
|---|---|---|
| 1 | B | A |
| 2 | C | B |
| 3 | C | A |
| 4 | — | A |
| 5 | A | A |
| 6 | — | C |
| 7 | — | A |
| 8 | B | A |
| 9 | — | C |
| 10 | — | B |
| 11 | A | A |
| 12 | A | A |
| 13 | A | A |
| 14 | A | A |
| 15 | A | A |
| 16 | A | B |
| 17 | A | B |
| 18 | — | C |
| 19 | A | C |
| 20 | C | C |

What is claimed is:

1. A compound of formula (I) or a pharmaceutically acceptable salt thereof: 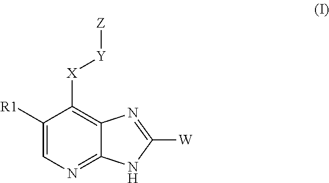
wherein,
R1 is hydrogen, halogen or CN;
X is O, NH, CH₂, S, SO or SO₂;
Y is phenyl or pyridyl;
Z is 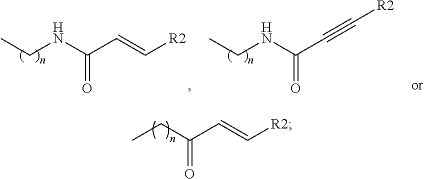
n is an integer ranging from 0 to 4;
R2 is each independently hydrogen, C₁₋₆alkoxy or di(C₁₋₆alkyl)aminomethyl; and
W is phenyl or pyridyl substituted with one or more substituents selected from the group consisting of hydrogen, halogen, hydroxy, nitro, C₁₋₆alkoxy, C₁₋₆alkoxycarbonyl, amino, C₁₋₆alkylamino, C₁₋₆alkylheterocycleamino, carbamoyl, C₁₋₆alkylcarbamoyl, di(C₁₋₆alkyl)carbamoyl, C₁₋₆alkylheterocyclecarbamoyl, C₁₋₆alkylheterocycleC₁₋₆alkyl, sulfamoyl, C₁₋₆alkylsulfanyl, C₁₋₆alkylsulfonyl, C₁₋₆alkylsulfinyl, C₁₋₆alkoxyC₁₋₆alkyl, C₁₋₆alkoxyC₁₋₆alkoxy, di(C₁₋₆alkyl)amino, di(C₁₋₆alkyl)aminoC₁₋₆alkyl, di(C₁₋₆alkyl)aminoC₁₋₆alkoxy, carboxyl, heterocycle, C₁₋₆alkylheterocycle, hydroxyheterocycle, hydroxyC₁₋₆alkylheterocycle, C₁₋₆alkoxyC₁₋₆alkylheterocycle, heterocyclic-oxy, heterocyclicC₁₋₆alkyl, heterocyclicaminoC₁₋₆alkyl, heterocycliccarbonyl, and heterocyclic-C₁₋₆alkylcarbonyl, wherein the heterocycle is independently a saturated 3- to 8-membered monocyclic hetero ring containing one or more of heteroatoms independently selected from N, O and S.

2. The compound of formula (I) or a pharmaceutically acceptable salt thereof of claim 1, wherein W may be selected from the group consisting of:

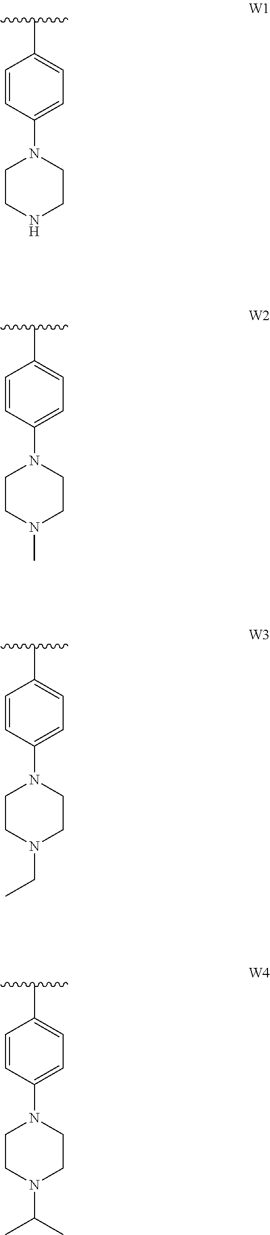 [SMILES: *c1ccc(N2CCNCC2)cc1 ; *c1ccc(N2CCN(C)CC2)cc1 ; *c1ccc(N2CCN(CC)CC2)cc1 ; *c1ccc(N2CCN(C(C)C)CC2)cc1]

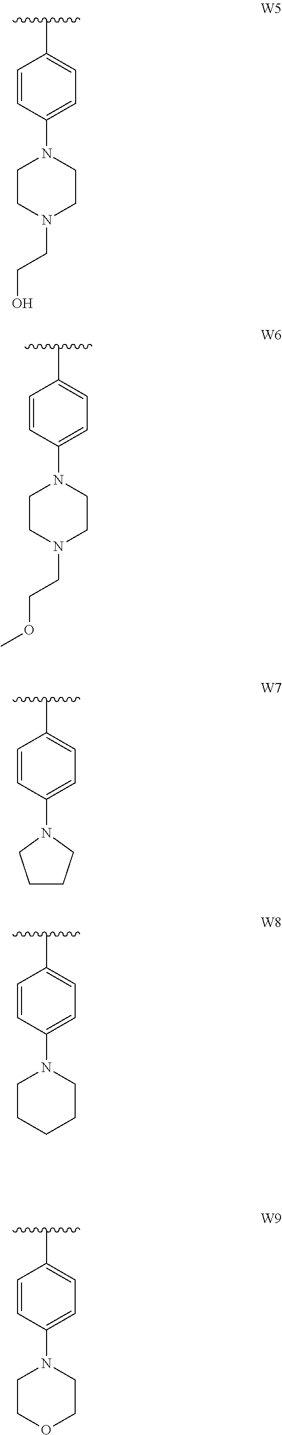 [SMILES: *c1ccc(N2CCN(CCO)CC2)cc1 ; *c1ccc(N2CCN(CCOC)CC2)cc1 ; *c1ccc(N2CCCC2)cc1 ; *c1ccc(N2CCCCC2)cc1 ; *c1ccc(N2CCOCC2)cc1]

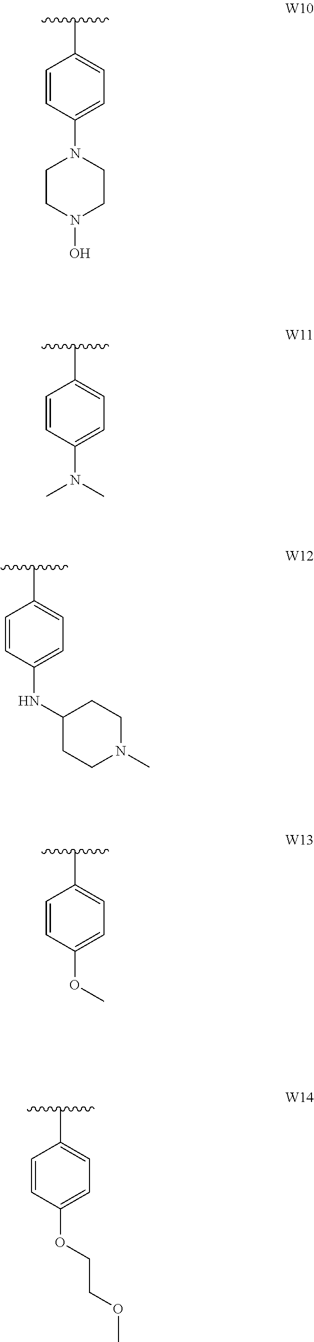

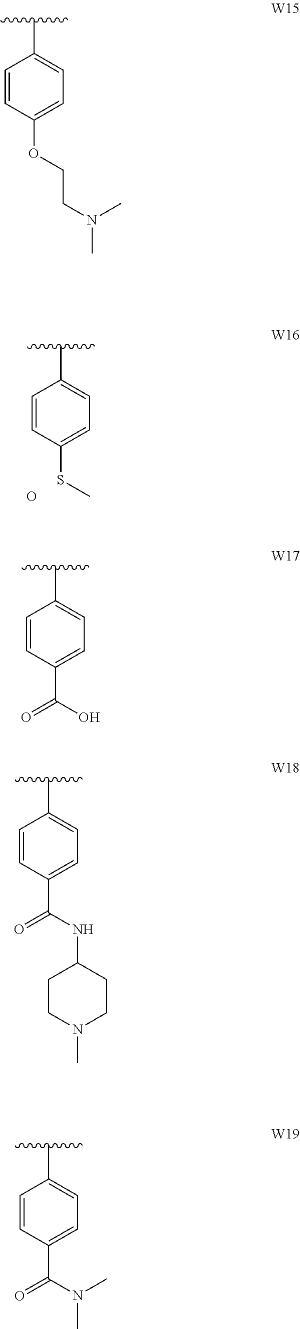

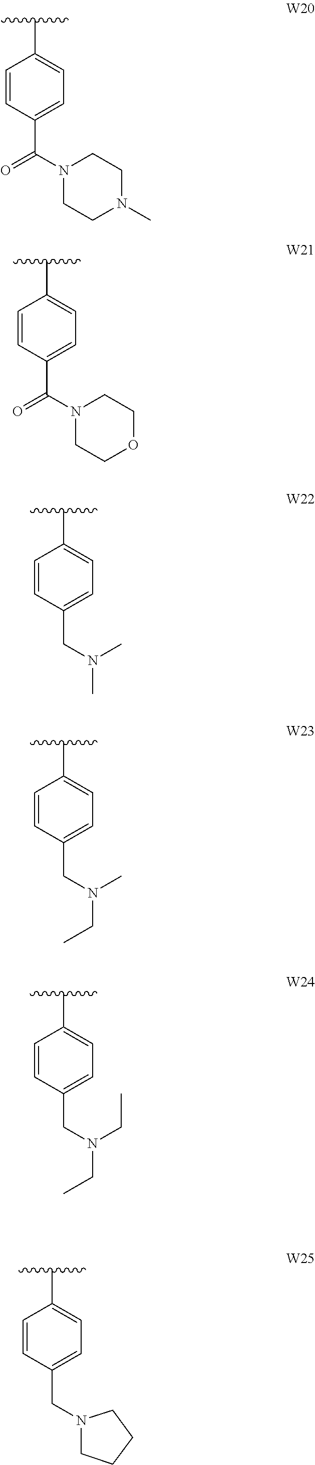 [SMILES: *c1ccc(C(=O)N2CCN(C)CC2)cc1 ; *c1ccc(C(=O)N2CCOCC2)cc1 ; *c1ccc(CN(C)C)cc1 ; *c1ccc(CN(C)CC)cc1 ; *c1ccc(CN(CC)CC)cc1 ; *c1ccc(CN2CCCC2)cc1]

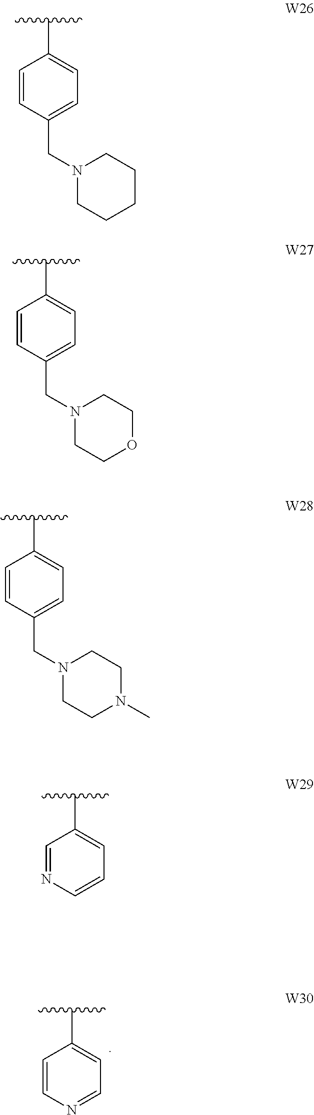 [SMILES: *c1ccc(CN2CCCCC2)cc1 ; *c1ccc(CN2CCOCC2)cc1 ; *c1ccc(CN2CCN(C)CC2)cc1 ; *c1cccnc1 ; *c1ccncc1]

3. The compound of formula (I) or a pharmaceutically acceptable salt thereof of claim 1, wherein the compound is selected from the group consisting of:
N-(3-(6-chloro-2-(4-(4-methylpiperazin-1-yl)phenyl)-3H-imidazo[4,5-b]pyridin-7-yloxy)phenyl)acrylamide;
N-(3-(6-chloro-2-(4-(4-hydroxypiperidin-1-yl)phenyl)-3H-imidazo[4,5-b]pyridin-7-yloxy)phenyl)acrylamide;
N-(3-(6-chloro-2-(4-(4-methylpiperazin-1-carbonyl)phenyl)-3H-imidazo[4,5-b]pyridin-7-yloxy)phenyl)acrylamide;
N-(3-(6-chloro-2-(4-(morpholin-4-carbonyl)phenyl)-3H-imidazo[4,5-b]pyridin-7-yloxy)phenyl)acrylamide;
N-(3-(6-chloro-2-(4-(1-methylpiperidin-4-ylamino)phenyl)-3H-imidazo[4,5-b]pyridin-7-yloxy)phenyl)acrylamide;
N-(3-(6-chloro-2-(4-(dimethylamino)phenyl)-3H-imidazo[4,5-b]pyridin-7-yloxy)phenyl)acrylamide;
N-(3-(6-chloro-2-(4-(methylsulphinyl)phenyl)-3H-imidazo[4,5-b]pyridin-7-yloxy)phenyl)acrylamide;
4-(7-(3-acrylamidophenoxy)-6-chloro-3H-imidazo[4,5-b]pyridin-2-yl)-N,N-dimethylbenzamide;
4-(7-(3-acrylamidophenoxy)-6-chloro-3H-imidazo[4,5-b]pyridin-2-yl)-benzoic acid;
4-(7-(3-acrylamidophenoxy)-6-chloro-3H-imidazo[4,5-b]pyridin-2-yl)-N-(1-methylpiperidin-4-yl)benzamide;
N-(3-(6-chloro-2-(4-((dimethylamino)methyl)phenyl)-3H-imidazo[4,5-b]pyridin-7-yloxy)phenyl)acrylamide;
N-(3-(6-chloro-2-(4-((diethylamino)methyl)phenyl)-3H-imidazo[4,5-b]pyridin-7-yloxy)phenyl)acrylamide;
N-(3-(6-chloro-2-(4-((ethyl(methyl)amino)methyl)phenyl)-3H-imidazo[4,5-b]pyridin-7-yloxy)phenyl)acrylamide;
N-(3-(6-chloro-2-(4-(pyrroldin-1-ylmethyl)phenyl)-3H-imidazo[4,5-b]pyridin-7-yloxy)phenyl)acrylamide;
N-(3-(6-chloro-2-(4-(piperidin-1-ylmethyl)phenyl)-3H-imidazo[4,5-b]pyridin-7-yloxy)phenyl)acrylamide;
N-(3-(6-chloro-2-(4-(morpholinomethyl)phenyl)-3H-imidazo[4,5-b]pyridin-7-yloxy)phenyl)acrylamide;
N-(3-(6-chloro-2-(4-((4-methylpiperazin-1-yl)methyl)phenyl)-3H-imidazo[4,5-b]pyridin-7-yloxy)phenyl)acrylamide;
N-(3-(6-chloro-2-(4-methoxyphenyl)-3H-imidazo[4,5-b]pyridin-7-yloxy)phenyl)acrylamide;
N-(3-(6-chloro-2-(pyridin-4-yl)-3H-imidazo[4,5-b]pyridin-7-yloxy)phenyl)acrylamide; and
N-(3-(2-(4-(4-methylpiperazin-1-yl)phenyl)-3H-imidazo[4,5-b]pyridin-7-yloxy)phenyl)acrylamide.

4. A pharmaceutical composition comprising the compound of formula (I) or a pharmaceutically acceptable salt thereof of claim 1 as an active ingredient for prevention or treatment of inflammatory diseases, autoimmune diseases, proliferative diseases or hyperproliferative diseases, immunologically mediated diseases, cancers or tumors.

5. The pharmaceutical composition of claim 4, wherein the inflammatory diseases, autoimmune diseases, proliferative diseases or hyperproliferative diseases, immunologically mediated diseases, cancers and tumors are mediated by one or more of kinases selected from the group consisting of: Janus kinase 3 (JAK3), Bruton's tyrosine kinase (BTK), IL-2 inducing T-cell kinase (ITK), resting lymphocyte kinase (RLK) and bone marrow tyrosine kinase (BMX).

6. The pharmaceutical composition of claim 4, wherein the inflammatory disease, autoimmune disease, proliferative diseases or hyperproliferative diseases, immunologically mediated diseases, cancers or tumors are mediated by aberrantly activated T-lymphocytes, B-lymphocytes or both.

7. The pharmaceutical composition of claim 4, wherein the inflammatory diseases, autoimmune diseases, proliferative diseases or hyperproliferative diseases, or immunologically mediated disease are selected from the group consisting of arthritis, rheumatoid arthritis, spondyloarthropathy, gouty arthritis, osteoarthritis, juvenile arthritis, other arthritic conditions, lupus, systemic lupus erythematosus (SLE), skin-related diseases, psoriasis, eczema, dermatitis, atopic dermatitis, pain, pulmonary disorder, lung inflammation, adult respiratory distress syndrome (ARDS), pulmonary sarcoidosis, chronic pulmonary inflammatory disease, chronic obstructive pulmonary disease (COPD), cardiovascular disease, artherosclerosis, myocardial infarction, congestive heart failure, cardiac reperfusion injury, inflammatory bowel disease, Crohn's disease, ulcerative colitis, irritable bowel syndrome, asthma, Sjogren's syndrome, autoimmune thyroid disease, urticaria, multiple sclerosis, scleroderma, allograft rejection, xenotransplantation, idiopathic thrombocytopenic purpura (ITP), Parkinson's disease, Alzheimer's disease, diabetic associated disease, inflammation, pelvic inflammatory disease, allergic rhinitis, allergic bronchitis, allergic sinusitis, leukemia, lymphoma, B-cell lymphoma, T-cell lymphoma, myeloma, acute lymphoid leukemia (ALL), chronic lymphoid leukemia (CLL), acute myeloid leukemia (AML), chronic myeloid leukemia (CML), hairy cell leukemia, Hodgkin's disease, non-Hodgkin's lymphoma, multiple myeloma, myelodysplastic syndrome (MDS), myeloproliferative neoplasms (MPN), diffuse large B-cell lymphoma and follicular lymphoma.

8. The pharmaceutical composition of claim 4, which further comprises any anticancer agents selected from the group consisting of cell signaling inhibitors, mitotic inhibitors, alkylating agents, antimetabolites, intercalating agents, topoisomerase inhibitors, immunotherapeutic agents, antihormonal agents and a mixture thereof.

9. The pharmaceutical composition of claim 4, which further comprises additional drugs selected from the group consisting of steroids, methotrexate, lefluonomide, anti-TNF a agents, calcineurin inhibitors, antihistamines and a mixture thereof.

10. A method for preventing or treating inflammatory diseases, autoimmune diseases, proliferative diseases or hyperproliferative diseases, immunologically mediated diseases, cancers or tumors in an mammal, comprising the step of administering to the mammal an effective amount of the compound of formula (I) of claim 1 or a pharmaceutically acceptable salt thereof.
